SEQUENCE LISTING (16 sequences)

SEQ ID NO: 1 - protein, 521 aa, Homo sapiens
MLAKGLPPRSVLVKGYQTFLSAPREGLGRLRVPTGEGAGISTRSPRPFNEIPSPGDNGWLNLYHFWRETGTHKVHLHHVQNFQKYGPIYREKLGNVESVYVIDPEDVALLFKSEGPNPERFLIPPWVAYHQYYQRPIGVLLKKSAAWKKDRVALNQEVMAPEATKNFLPLLDAVSRDFVSVLHRRIKKAGSGNYSGDISDDLFRFAFESITNVIFGERQGMLEEVVNPEAQRFIDAIYQMFHTSVPMLNLPPDLFRLFRTKTWKDHVAAWDVIFSKADIYTQNFYWELRQKGSVHHDYRGMLYRLLGDSKMSFEDIKANVTEMLAGGVDTTSMTLQWHLYEMARNLKVQDMLRAEVLAARHQAQGDMATMLQLVPLLKASIKETLRLHPISVTLQRYLVNDLVLRDYMIPAKTLVQVAIYALGREPTFFFDPENFDPTRWLSKDKNITYFRNLGFGWGVRQCLGRRIAELEMTIFLINMLENFRVEIQHLSDVGTTFNLILMPEKPISFTFWPFNQEATQQ

SEQ ID NO: 2 - DNA, 20 bp, Artificial Sequence
  CYP11A1 cDNA sequence position 1684-1703
tcgtctgtcccaagtcctgc

SEQ ID NO: 3 - DNA, 22 bp, Artificial Sequence
  CYP11A1 cDNA sequence at position 1728-1749
aggttgaatgggttctcagtgg

SEQ ID NO: 4 - DNA, 20 bp, Artificial Sequence
  Primer for CYP11A1
tcgtctgtcccaagtcctgc

SEQ ID NO: 5 - DNA, 22 bp, Artificial Sequence
  Primer for CYP11A1
ccactgagaacccattcaacct

SEQ ID NO: 6 - DNA, 20 bp, Artificial Sequence
  Primer for cycophilin B
actgaagcactacgggcctg

SEQ ID NO: 7 - DNA, 19 bp, Artificial Sequence
  Primer for cyclophilin B
agccgttggtgtctttgcc

SEQ ID NO: 8 - DNA, 20 bp, Artificial Sequence
  Primer for the ribosomal protein S9
ggtcaaatttaccctggcca

SEQ ID NO: 9 - DNA, 22 bp, Artificial Sequence
  Primer for the ribosomal protein S9
tctcatcaagcgtcagcagttc

SEQ ID NO: 10 - DNA, 19 bp, Artificial Sequence
  Primer for beta-actin
tggaacggtgaaggtgaca

SEQ ID NO: 11 - DNA, 19 bp, Artificial Sequence
  Primer for beta-actin
ggcaagggacttcctgtaa

SEQ ID NO: 12 - DNA, 20 bp, Artificial Sequence
  Primer for GAPDH
cgtcatgggtgtgaaccatg

SEQ ID NO: 13 - DNA, 21 bp, Artificial Sequence
  Primer for GAPDH
gctaagcagttggtggtgcag

SEQ ID NO: 14 - DNA, 21 bp, Artificial Sequence
  Primer for the transferrin receptor
gtcgctggtcagttcgtgatt

SEQ ID NO: 15 - DNA, 23 bp, Artificial Sequence
  Primer for the transferrin receptor
agcagttggctgttgtacctctc

SEQ ID NO: 16 - DNA, 22 bp, Artificial Sequence
  RT PCR primer for CYP11A1
aggttgaatgggttctcagtgg

(19) United States
(12) Patent Application Publication
(10) Pub. No.: US 2006/0099585 A1
(43) Pub. Date: May 11, 2006

(54) Diagnostic and therapeutic use of cyp11a1 for neurodegenerative diseases

(75) Inventors: Thomas Hesterkamp, HAMBURG (DE); Heinz Von Der Kammer, Hamburg (DE); Ralf Krappa, Tornesch (DE); Johannes Pohlner, Hamburg (DE)

Correspondence Address: STERNE, KESSLER, GOLDSTEIN & FOX PLLC, 1100 NEW YORK AVENUE, N.W., WASHINGTON, DC, 20005, US

(73) Assignee: EVOCTEC NEUROSCIENCES GMBH, HAMBURG (DE)

(21) Appl. No.: 10/515,598

(22) PCT Filed: May 28, 2003
(86) PCT No.: PCT/EP03/05622

§ 371 Date: Jul. 29, 2005

Related U.S. Application Data
(60) Provisional application No. 60/383,105, filed on May 28, 2002.

(30) Foreign Application Priority Data
May 28, 2002 (EP) 02011818.8

(51) Int. Cl. C12Q 1/68; A01K 67/027

(52) U.S. Cl. 435/6; 800/12

(57) ABSTRACT

The present invention discloses the differential expression of the CYP11A1 gene in specific brain regions of Alzheimer's disease patients. Based on this finding, this invention provides a method for diagnosing or prognosticating a neurodegenerative disease, in particular Alzheimer's disease in a subject, or for determining whether a subject is at increased risk of developing such a disease. Furthermore, this invention provides therapeutic and prophylactic methods for treating or preventing Alzheimer's disease and related neurodegenerative disorders using the CYP11A1 gene and gene products. A method of screening for modulating agents of neurodegenerative diseases is also disclosed.

The present invention relates to methods of diagnosing, prognosticating and monitoring the progression of neurodegenerative diseases in a subject. Furthermore, methods of therapy control and screening for modulating agents of neurodegenerative diseases are provided. The invention also discloses pharmaceutical compositions, kits, and recombinant animal models.

Neurodegenerative diseases, in particular Alzheimer's disease (AD), have a strongly debilitating impact on a patient's life. Furthermore, these diseases constitute an enormous health, social, and economic burden. AD is the most common neurodegenerative disease, accounting for about 70% of all dementia cases, and it is probably the most devastating age-related neurodegenerative condition affecting about 10% of the population over 65 years of age and up to 45% over age 85 (for a recent review see Vickers et al., Progress in Neurobiology 2000, 60: 139-165). Presently, this amounts to an estimated 12 million cases in the US, Europe, and Japan. This situation will inevitably worsen with the demographic increase in the number of old people (“aging of the baby boomers”) in developed countries. The neuropathological hallmarks that occur in the brains of individuals with AD are senile plaques, composed of amyloid-β protein, and profound cytoskeletal changes coinciding with the appearance of abnormal filamentous structures and the formation of neurofibrillary tangles.

The amyloid-β (Aβ) protein evolves from the cleavage of the amyloid precursor protein (APP) by different kinds of proteases. The cleavage by the β/γ-secretase leads to the formation of Aβ peptides of different lengths, typically a short more soluble and slow aggregating peptide consisting of 40 amino acids and a longer 42 amino acid peptide, which rapidly aggregates outside the cells, forming the characteristic amyloid plaques (Selkoe, Physiological Rev 2001, 82: 741-66; Greenfield et al,. Frontiers Bioscience 2000, 5: D72-83). Two types of plaques, diffuse plaques and neuritic plaques, can be detected in the brain of AD patients, the latter ones being the classical, most prevalent type. They are primarily found in the cerebral cortex and hippocampus. The neuritic plaques have a diameter of 50 μm to 200 μm and are composed of insoluble fibrillar amyloids, fragments of dead neurons, of microglia and astrocytes, and other components such as neurotransmitters, apolipoprotein E, glycosaminoglycans, α1-antichymotrypsin and others. The generation of toxic Aβ deposits in the brain starts very early in the course of AD, and it is discussed to be a key player for the subsequent destructive processes leading to AD pathology. The other pathological hallmarks of AD are neurofibrillary tangles (NFTs) and abnormal neurites, described as neuropil threads (Braak and Braak, Acta Neuropathol 1991, 82: 239-259). NFTs emerge inside neurons and consist of chemically altered tau, which forms paired helical filaments twisted around each other. Along the formation of NFTs, a loss of neurons can be observed. It is discussed that said neuron loss may be due to a damaged microtubule-associated transport system (Johnson and Jenkins, J Alzheimers Dis 1996, 1: 38-58; Johnson and Hartigan, J Alzheimers Dis 1999, 1: 329-351). The appearance of neurofibrillary, tangles and their increasing number correlates well with the clinical severity of AD (Schmitt et al., Neurology 2000, 55: 370-376).

AD is a progressive disease that is associated with early deficits in memory formation and ultimately leads to the complete erosion of higher cognitive function. The cognitive disturbances include among other things memory impairment, aphasia, agnosia and the loss of executive functioning. A characteristic feature of the pathogenesis of AD is the selective vulnerability of particular brain regions and subpopulations of nerve cells to the degenerative process. Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10: 184-92).

The age of onset of AD may vary within a range of 50 years, with early-onset AD occurring in people younger than 65 years of age, and late-onset of AD occurring in those older than 65 years. About 10% of all AD cases suffer from early-onset AD, with only 1-2% being familial, inherited cases.

Currently, there is no cure for AD, nor is there an effective treatment to halt the progression of AD or even to diagnose AD ante-mortem with high probability. Several risk factors have been identified that predispose an individual to develop AD, among them most prominently the epsilon 4 allele of the three different existing alleles (epsilon 2, 3, and 4) of the apolipoprotein E gene (ApoE) (Strittmatter et al., Proc Natl Acad Sci USA 1993, 90: 1977-81; Roses, Ann NY Acad Sci 1998, 855: 738-43). Efforts to detect further susceptibility genes and disease-linked polymorphisms lead to the assumption that specific regions and genes on human chromosomes 10 and 12 may be associated with late-onset AD (Myers et al., Science 2000, 290: 2304-5; Bertram et al., Science 2000, 290: 2303; Scott et al., Am J Hum Genet 2000, 66: 922-32).

Although there are rare examples of early-onset AD which have been attributed to genetic defects in the genes for amyloid precursor protein (APP) on chromosome 21, presenilin-1 on chromosome 14, and presenilin-2 on chromosome 1, the prevalent form of late-onset sporadic AD is of hitherto unknown etiologic origin. The mutations found to date account for only half of the familial AD cases, which is less than 2% of all AD patients. The late onset and complex pathogenesis of neurodegenerative disorders pose a formidable challenge to the development of therapeutic and diagnostic agents. It is crucial to expand the pool of potential drug targets and diagnostic markers. It is therefore an object of the present invention to provide insight into the pathogenesis of neurological diseases and to provide methods, materials, agents, compositions, and animal models which are suited inter alia for the diagnosis and development of a treatment of these diseases. This object has been solved by the features of the independent claims. The subclaims define preferred embodiments of the present invention.

The brain is a target site of peripheral steroid hormones synthesized and secreted into circulation by the adrenal glands, gonads and the feto-placental axis. It is well-known, for instance, that gonadal androgens act on the brain to influence the reproductive behavior of individuals. However, the brain is also a site of synthesis of steroid hormones, a process termed neuro-steroidogenesis. Only two decades have passed from the first description of dehydroepiandrosterone sulfate production in rodent brain (Corpechot et al., Proc. Natl. Acad. Sci. U.S.A. 1981, 78:4704-4707). Therefore, this field of research is a young discipline at the interface of neurology, endocrinology, and behavioral sciences.

There are two principle categories of steroid hormone action in brain tissue, a genomic action and an immediate non-genomic action. At the genomic level, steroid hormones bind to intracellular/nuclear hormone receptors that regulate transcription of target genes. These adaptive responses generally occur within hours or days. In addition, steroid hormones show an immediate impact on the excitability of the central nervous system through action on neurotransmitter-gated ion channels (for review: Stoffel-Wagner, Eur. J. Endocrinol. 2001, 145:669-679). In particular, pregnenolone sulfate potentiates the calcium conductivity of N-methyl-D-aspartate receptors and suppresses the chloride conductivity of the gamma-aminobutyric acid receptors in rat hippocampal neurons, thus affecting the overall neuronal excitability (Irwin et al., Neurosci. Lett. 1992, 141:30-34; Harrison et al., J. Neurosci. 1987, 7: 604-609).

In order to understand the physiological roles of neurosteroidogenesis in the mammalian brain it is important to identify the brain regions involved and the type(s) of cells that are proficient for the key enzymes and therefore likely to be active in the synthetic process. Most of the current knowledge in this regard is derived from studies in rodents, and the overall picture that emerges is that glial cells, oligodendrocytes and astrocytes play a major role in neurosteroid formation and metabolism in cerebrum and cerebellum (for review: Tsutsui et al., Neurosci. Res. 2000, 36:261-273). On the other hand, in more recent studies in the rat system, pyramidal and granule neurons of the hippocampus (Furukawa et al., J. Neurochem. 1998, 71:2231-2238; Kimoto et al., Endocrinology 2001, 142:3578-3589) and Purkinje cell neurons of the cerebellum (Ukena et al., Endocrinology 1998, 139: 137-147; Ukena et al., Endocrinology 1999, 140: 805-813) were shown to possess the key enzymes of neurosteroidogenesis as well. Due to the difficulty of obtaining fresh brain material, analogous studies in the human system are in their infancy. Using a sensitive reverse transcriptase polymerase chain reaction approach, the messenger RNA for the rate-limiting enzyme of neurosteroidogenesis, CYP11A1 (alias: cytochrome P450 11A1, cholesterol side chain cleavage enzyme, P450-scc, cholesterol 20-22-desmolase, steroid 20-22 lyase, cholesterol monooxygenase), was detected in surgical specimen of human brain (Watzka et al., J. Neuroendocrinol. 1999, 11:901-905). CYP11A1 mRNA was found in the cortex of temporal and frontal lobes and hippocampus, albeit at levels 200 times lower than in the adrenal gland. The authors detected sex- and age-specific differences in CYP11A1 mRNA. Messenger RNAs for two other key enzymes of neurosteroidogenesis, CYP21 and 17-beta hydroxysteroid dehydrogenase, were also detected in surgical specimen of the temporal lobe and hippocampus (Beyenburg et al., Neurosci. Lett. 2001, 308:111-114; Stoffel-Wagner et al., J. Endocrinol. 1999, 160:119-126). Based on this small complement of enzymes, the following steroids can principally be synthesized in situ: 20-alpha-hydroxycholesterol, 22-beta-hydroxy-cholesterol, 20-alpha-22-beta-dihydroxy-cholesterol, pregnenolone, 21-hydroxypregnenolone, progesterone, and 11-deoxycorticosterone. However, additional neurosteroidogenic enzymes have been detected in brain, including CYP19 (aromatase), 5-alpha-reductase, CYP17, 3-alpha-hydroxysteroid dehydrogenase, CYP11B1 and others (for review: Stoffel-Wagner, Eur. J. Endocrinol. 2001, 145:669-679) and, therefore, many more neurosteroids are likely to be synthesized within the brain.

The potential (patho)physiological implications of the above findings remain to be elucidated. For instance, the anticonvulsive, anesthetic, and anxiolytic effects of neuroactive steroids are attributed to their capacity to modulate the gamma-aminobutyric acid receptor function and are therefore of immediate, non-genomic nature (Stoffel-Wagner, Eur. J. Endocrinol. 2001, 145:669-679). Furthermore, it has been postulated that neurosteroids act on nerve cells through membrane receptors coupled to G proteins, various neuropeptide receptors and progesterone receptors (Orchinik et al., Proc. Natl. Acad. Sci. U.S.A. 1992, 89:3830-3834; Grazzini et al., Nature 1998, 392:509-512; Rupprecht et al., Neuron 1993, 11:523-530). On a more phenomenological level, the abundant neurosteroids dehydroepiandrosterone and dehydro-epiandrosterone sulfate, generated from pregnenolone by action of cytochrome P450 17-alpha-lyase and hydroxysteroid sulfotransferase, are discussed as neuroprotective agents, whose levels decrease with age and under stress (Kimonides et al., Proc. Natl. Acad. Sci. U.S.A. 1998, 95:1852-1857). In fact, a potential role of pregnenolone, dehydroepiandrosterone and their sulfate esters in learning, memory, and cognitive aging is under controversial discussion (Vallee et al., Brain Research Reviews 2001, 37:301-312; international patent application WO01/55692). Fluctuations in neuroactive steroids are thought to contribute to the increased risk of developing psychiatric diseases in women during the perimenstrual phase, pregnancy, the post partum period, and menopause. On the cognitive level, estrogens and androgens impact on verbal fluency, the performance of spatial tasks, verbal memory tests, and fine rotor skills (for review and further physiological and pathophysiological implications of neurosteroid action on the central nervous system: McEwen, Annu. Rev. Neurosci. 1999, 22:105-122; Stoffel-Wagner, Eur. J. Endocrinol. 2001, 145:669-679).

The instant invention discloses the differential expression of the CYP11A1 gene in different regions of the AD brain but not in the brain of healthy age-matched control individuals. CYP11A1 (alias: cytochrome P450 11A1, cholesterol side chain cleavage enzyme, P450-scc, cholesterol 20-22-desmolase, steroid 20-22 lyase, cholesterol monooxygenase) is encoded by the synonymous gene CYP11A1 on human chromosome 15q23-q24 (Chung et al., Proc. Natl. Acad. Sci. U.S.A. 1986, 83:8962-8966; GenBank accession number M14565; U.S. Pat. No. 5,045,471). Mature human CYP11A1 is a 482 amino acids protein localized to the inner mitochondrial membrane and synthesized as a 521 amino acids precursor protein on cytoplasmic ribosomes (GenBank accession number P05108). CYP11A1 belongs to the cytochrome P450 family of monooxygenases and catalyzes the synthesis of pregnenolone from cholesterol, the first and committed step of steroid hormone synthesis. This reaction is also known as cholesterol side chain cleavage and entails three enzymatic sub-steps. The highest expression levels of CYP11A1 are found in the adrenal cortex, testis, ovary, and placenta. Recently, an insertion mutation in CYP11A1 was described that was phenotypically associated with a disease condition named lipoid congenital adrenal hyperplasia, thus stressing the prominent role of CYP11A1 in adrenal steroidogenesis (Tajima et al., J. Clin. Endocr. Metab. 2001, 86:3820-3825). To date, no mutations in CYP11A1 gene have been described to be associated with neurodegenerative diseases. Likewise, no experiments have been described that demonstrate a relationship between the dysregulation of the CYP11A1 gene expression and the pathology of neurodegenerative diseases, in particular AD. The disclosures of the instant invention offer new ways, inter alia, for the diagnosis and treatment of said diseases.

The singular forms “a”, “an”, and “the” as used herein and in the claims include plural reference unless the context dictates otherwise. For example, “a cell” means as well a plurality of cells, and so forth. The term “and/or” as used in the present specification and in the claims implies that the phrases before and after this term are to be considered either as alternatives or in combination. For instance, the wording “determination of a level and/or an activity” means that either only a level, used or only an activity, a or both a level and an activity are determined. The term “level” as used herein is meant to comprise a gage of, or a measure of the amount of, or a concentration of a transcription product, for instance an mRNA, or a translation product, for instance a protein or polypeptide. The term “activity” as used herein shall be understood as a measure for the ability of a transcription product or a translation product to produce a biological effect or a measure for a level of biologically active molecules. The term “activity” also refers to enzymatic activity. The terms “level” and/or “activity” as used herein further refer to gene expression levels or gene activity. Gene expression can be defined as the utilization of the information contained in a gene by transcription and translation leading to the production of a gene product. “Dysregulation” shall mean an upregulation or downregulation of gene expression. A gene product comprises either RNA or protein and is the result of expression of a gene. The amount of a gene product can be used to measure how active a gene is. The term “gene” as used in the present specification and in the claims comprises both coding regions (exons) as well as non-coding regions (e.g. non-coding regulatory elements such as promoters or enhancers, introns, leader and trailer sequences). The term “ORF” is an acronym for “open reading frame” and refers to a nucleic acid sequence that does not possess a stop codon in at least one reading frame and therefore can potentially be translated into a sequence of amino acids. “Regulatory elements” shall comprise inducible and non-inducible promoters, enhancers, operators, and other elements that drive and regulate gene expression. The term “fragment” as used herein is meant to comprise e.g. an alternatively spliced, or truncated, or otherwise cleaved transcription product or translation product. The term “derivative” as used herein refers to a mutant, or an RNA-edited, or a chemically modified, or otherwise altered transcription product, or to a mutant, or chemically modified, or otherwise altered translation product. For instance, a “derivative” may be generated by processes such as altered phosphorylation, glycosylation, acetylation, lipidation, or altered signal peptide cleavage or other types of maturation cleavage. These processes may occur post-translationally. The term “modulator” as used in the present invention and in the claims refers to a molecule capable of changing or altering the level and/or the activity, of a gene, or a transcription product of a gene, or a translation product of a gene. Preferably, a “modulator” is capable of changing or altering the biological activity of a transcription product or a translation product of a gene. Said modulation, for instance, may be an increase or a decrease in enzyme activity, a change in binding characteristics, or any other change or alteration in the biological, functional, or immunological properties of said translation product of a gene. The terms “agent”, “reagent”, or “compound” refer to any substance, chemical, composition or extract that have a positive or negative biological effect on a cell, tissue, body fluid, or within the context of any biological system, or any assay system examined. They can be agonists, antagonists, partial agonists or inverse agonists of a target. Such agents, reagents, or compounds may be nucleic acids, natural or synthetic peptides or protein complexes, or fusion proteins. They may also be antibodies, organic or anorganic molecules or compositions, small molecules, drugs and any combinations of any of said agents above. They may be used for testing, for diagnostic or for therapeutic purposes. The terms “oligonucleotide primer” or “primer” refer to short nucleic acid sequences which can anneal to a given target polynucleotide by hybridization of the complementary base pairs and can be extended by a polymerase. They may be chosen to be specific to a particular sequence or they may be randomly selected, e.g. they will prime all possible sequences in a mix. The length of primers used herein may preferably vary from 10 nucleotides to 80 nucleotides. “Probes” are short nucleic acid sequences of the nucleic acid sequences described and disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample. As used herein, “homolog or homology” is a term used in the art to describe the relatedness of a nucleotide or peptide sequence to another nucleotide or peptide sequence; which is determined by the degree of identity and/or similarity between said sequences compared. The term “variant” as used herein refers to any polypeptide or protein, in reference to polypeptides and proteins disclosed in the present invention, in which one or more amino acids are added and/or substituted and/or deleted and/or inserted at the N-terminus, and/or the C-terminus, and/or within the native amino acid sequences of the native polypeptides or proteins of the present invention. Furthermore, the term “variant” shall include any shorter or longer version of a polypeptide or protein. “Variants” shall also comprise sequences that have at least about 80% sequence identity, more preferably at least about 90% sequence identity, and most preferably at least about 95% sequence identity with the amino acid sequence of the human CYP11A1 gene, SEQ ID NO. 1. “Variants” of a protein molecule include, for example, proteins with conservative amino acid substitutions in highly conservative regions. “Proteins and polypeptides” of the present invention include variants, fragments and chemical derivatives of the protein comprising the amino acid sequence of CYP11A1, SEQ ID NO. 1. They can include proteins and polypeptides which can be isolated from nature or be produced by recombinant and/or synthetic means. Native proteins or polypeptides refer to naturally-occurring truncated or secreted forms, naturally occurring variant forms (e.g. splice-variants) and naturally occurring allelic variants. The term “isolated” as used herein is considered to refer to molecules that are removed from their natural environment, i.e. isolated from a cell or from a living organism in which they normally occur, and that are separated or essentially purified from the coexisting components with which they are found to be associated in nature. This notion further means that the sequences encoding such molecules can be linked by the hand, of man to polynucleotides, to which they are not linked in their natural state, and that such molecules can be produced by recombinant and/or synthetic means. Even if for said purposes those sequences may be introduced into living or non-living organisms by methods known to those skilled in the art, and even if those sequences are still present in said organisms, they are still considered to be isolated. In the present invention, the terms “risk”, “susceptibility”, and “predisposition” are tantamount and are used with respect to the probability of developing a neurodegenerative disease, preferably Alzheimer's disease.

The term ‘AD’ shall mean Alzheimer's disease. “AD-type neuropathology” as used herein refers to neuropathological, neurophysiological, histopathological and clinical hallmarks as described in the instant invention and as commonly known from state-of-the-art literature (see: Iqbal, Swaab, Winblad and Wisniewski, Alzheimer's Disease and Related Disorders (Etiology, Pathogenesis and Therapeutics), Wiley & Sons, New York, Weinheim, Toronto, 1999;; Scinto and Daffner, Early Diagnosis of Alzheimer's Disease, Humana Press, Totowa, N.J., 2000; Mayeux and Christen, Epidemiology of Alzheimer's Disease: From Gene to Prevention, Springer Press, Berlin, Heidelberg, N.Y., 1999; Younkin, Tanzi and Christen, Presenilins and Alzheimer's Disease, Springer Press, Berlin, Heidelberg, N.Y., 1998). Neurodegenerative diseases or disorders according to the present invention comprise Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia and other tauopathies, and mild-cognitive impairment. Further conditions involving neurodegenerative processes are, for instance, age-related macular degeneration, narcolepsy, motor neuron diseases, prion diseases, traumatic nerve injury and repair, and multiple sclerosis.

In one aspect, the invention features a method of diagnosing or prognosticating a neurodegenerative disease in a subject, or determining whether a subject is at increased risk of developing said disease. The method comprises: determining a level, or an activity, or both said level and said activity of (I) a transcription product of the CYP11A1 gene, and/or of (ii) a translation product of the CYP11A1 gene, and/or of (iii) a fragment, or derivative, or variant of said transcription or translation product in a sample from said subject and comparing said level, and/or said activity to a reference value representing a known disease or health status, thereby diagnosing or prognosticating said neurodegenerative disease in said subject, or determining whether said subject is at increased risk of developing said neurodegenerative disease.

The invention also relates to the construction and the use of primers and probes which are unique to the nucleic acid sequences of the CYP11A1 gene, or fragments, or variants thereof, as disclosed in the present invention. The oligonucleotide primers and/or probes can be labeled specifically with fluorescent, bioluminescent, magnetic, or radioactive substances. The invention further relates to the detection and the production of said nucleic acid sequences, or fragments and/or variants thereof, using said specific oligonucleotide primers in appropriate combinations. PCR-analysis, a method well known to those skilled in the art, can be performed with said primer combinations to amplify said gene specific nucleic acid sequences from a sample, containing nucleic acids. Such sample may be derived either from healthy or diseased subjects. Whether an amplification results in a specific nucleic acid product or not, and whether a fragment of different length can be obtained or not, may be indicative for a neurodegenerative disease, in particular Alzheimer's disease. Thus, the invention provides nucleic acid sequences, oligonucleotide primers, and probes of at least 10 bases in length up to the entire coding and gene sequences, useful for the detection of gene mutations and single nucleotide polymorphisms in a given sample comprising nucleic acid sequences to be examined, which may be associated with neurodegenerative diseases, in particular Alzheimer's disease. This feature has utility for developing rapid DNA-based diagnostic tests, preferably also in the format of a kit.

In a further aspect, the invention features a method of monitoring the progression of a neurodegenerative disease in a subject. A level, or an activity, or both said level and said activity, of (i) a transcription product of the CYP11A1 gene, and/or of (ii) a translation product of the CYP11A1 gene, and/or of (iii) a fragment, or derivative, or variant of said transcription or translation product in a sample from said subject is determined. Said level and/or said activity is compared to a reference value representing a known disease or health status. Thereby the progression of said neurodegenerative disease in said subject is monitored.

In still a further aspect, the invention features a method of evaluating a treatment for a neurodegenerative disease, comprising determining a level, or an activity, or both said level and said activity of (i) a transcription product of the CYP11A1 gene, and/or of (ii) a translation product of the CYP11A1,gene, and/or of (iii) a fragment, or derivative, or variant of said transcription or translation product in a sample obtained from a subject being treated for said disease. Said level, or said activity, or both said level and said activity are compared to a reference value representing a known disease or health status, thereby evaluating the treatment for said neurodegenerative disease.

In a preferred embodiment of the herein claimed methods, kits, recombinant animals, molecules, assays, and uses of the instant invention, said gene coding for a human monooxygenase of the cytochrome P450 family, is the gene coding for human CYP11A1, also termed cytochrome P450 11A1, or cholesterol side chain cleavage enzyme, or P450-scc, or cholesterol 20-22-desmolase, or steroid 20-22 lyase, or cholesterol monooxygenase, represented by SEQ ID NO. 1 (GenBank accession number P05108; mRNA GenBank accession number M14565).

In a further preferred embodiment of the herein claimed methods, kits, recombinant animals, molecules, assays, and uses of the instant invention, said neurodegenerative disease or disorder is Alzheimer's disease, and said subjects, suffer from Alzheimer's disease.

The present invention discloses the differential expression and regulation of the CYP11A1 gene in specific brain regions of AD patients. Consequently, the CYP11A1 gene and its corresponding transcription and/or translation products may have a causative role in the regional selective neuronal degeneration typically observed in AD. Alternatively, CYP11A1 transcription and/or translation products may confer a neuroprotective function to the remaining surviving nerve cells. Based on these disclosures, the present invention has utility for the diagnostic evaluation and prognosis as well as for the identification of a predisposition to a neurodegenerative disease, in particular AD. Furthermore, the present invention provides methods for the diagnostic monitoring of patients undergoing treatment for such a disease.

It is particularly preferred that said sample to be analyzed and determined is selected from the group comprising brain tissue, or other tissues, organs, or body cells. The sample can also comprise cerebrospinal fluid or other body fluids including saliva, urine, blood, serum plasma, or mucus. Preferably, the methods of diagnosis, prognosis, monitoring the progression or evaluating a treatment for a neurodegenerative disease, according to the instant invention, can be practiced ex corpore, and such methods preferably relate to samples, for instance, body fluids or cells, removed, collected, or isolated from a subject or patient.

In further preferred embodiments, said reference value is that of a level, or an activity, or both said level and said activity of (i) a transcription product of the CYP11A1 gene, and/or of (ii) a translation product of the CYP11A1 gene, and/or of (iii) a fragment, or derivative, or variant of said transcription or translation product in a sample from a subject not suffering from said neurodegenerative disease.

In preferred embodiments, an alteration in the level and/or activity of a transcription product of the CYP11A1 gene and/or a translation product of the CYP11A1 gene, and/or a fragment, or derivative, or variant of said transcription or translation product, in a sample cell, or tissue, or body fluid from said subject relative to a reference value representing a known health status indicates a diagnosis, or prognosis, or increased risk of becoming diseased with a neurodegenerative disease, particularly AD.

In preferred embodiments, the measurement of the level of transcription products of the CYP11A1 gene is performed in a sample from a subject using a quantitative PCR-analysis with primer combinations to amplify said gene specific sequences from cDNA obtained by reverse transcription of RNA extracted from a sample of a subject. A Northern blot with probes specific for said gene can also be applied. It might further be preferred to measure transcription products by means of chip-based microarray technologies. These techniques are known to those of ordinary skill in the art (see Sambrook and Russell, Molecular Cloning: A Laboratory Manual, Cold Spring Harbor Laboratory Press, Cold Spring Harbor, N.Y., 2001; Schena M., Microarray Biochip Technology, Eaton Publishing, Natick, Mass., 2000). An example of an immunoassay is the detection and measurement of enzyme activity as disclosed and described in the patent application WO 02/14543.

Furthermore, a level and/or an activity of a translation product of the CYP11A1 gene and/or a fragment, or variant, or derivative of said translation product, and/or the level of activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). Enzymatic activity of a translation product of the CYP11A1 gene, in particular monooxygenase activity, may me measured by in vitro, cell-based, or in vivo assays. All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

In a preferred embodiment, the level, or the activity, or both said level and said activity of (i) a transcription product of the CYP11A1 gene, and/or of (ii) a translation product of the CYP11A1 gene, and/or of (iii) a fragment, or derivative, or variant of said transcription or translation product in a series of samples taken from said subject over a period of time is compared, in order to monitor the progression of said disease. In further preferred embodiments, said subject receives a treatment prior to one or more of said sample gatherings. In yet another preferred embodiment, said level and/or activity is determined before and after said treatment of said subject.

In another aspect, the invention features a kit for diagnosing or prognosticating neurodegenerative diseases, in particular AD, in a subject, or determining the propensity or predisposition of a subject to develop a neurodegenerative disease, in particular AD, said kit comprising:

(a) at least one reagent which is selected from the group consisting of (i) reagents that selectively detect a transcription product of the CYP11A1 gene (ii) reagents that selectively detect a translation product of the CYP11A1 gene; and

(b) an instruction for diagnosing, or prognosticating a neurodegenerative disease, in particular AD, or determining the propensity or predisposition of a subject to develop such a disease by

detecting a level, or an activity, or both said level and said activity, of said transcription product and/or said translation product of the CYP11A1 gene, in a sample from said subject; and
diagnosing or prognosticating a neurodegenerative disease, in particular AD, or determining the propensity or predisposition of said subject to develop such a disease,
   wherein a varied level, or activity, or both said level and said activity, of said transcription product and/or said translation product compared to a reference value representing a known health status; or a level, or activity, or both said level and said activity, of said transcription product and/or said translation product similar or equal to a reference value representing a known disease status, indicates a diagnosis or prognosis of a neurodegenerative disease, in particular AD, or an increased propensity or predisposition of developing such a disease. The kit, according to the present invention, may be particularly useful for the identification of individuals that are at risk of developing a neurodegenerative disease, in particular AD. Consequently, the kit, according to the invention, may serve as a means for targeting identified individuals for early preventive measures or therapeutic intervention prior to disease onset, before irreversible damage in the course of the disease has been inflicted. Furthermore, in preferred embodiments, the kit featured in the invention is useful for monitoring a progression of a neurodegenerative disease, in particular AD, in a subject, as well as monitoring success or failure of therapeutic treatment for such a disease of said subject.

In another aspect, the invention features a method of treating or preventing a neurodegenerative disease, in particular AD, in a subject comprising the administration to said subject in a therapeutically or prophylactically effective amount of an agent or agents which directly or indirectly affect a level, or an activity, or both said level and said activity, of (i) the CYP11A1 gene, and/or (ii) a transcription product of the CYP11A1 gene, and/or (iii) a translation product of the CYP11A1 gene, and/or (iv) a fragment, or variant, or derivative of (i) to (iii). Said agent may comprise a small molecule, or it may also comprise a peptide, an oligopeptide, or a polypeptide. Said peptide, oligopeptide, or polypeptide may comprise an amino acid sequence of a translation product of the CYP11A1 gene, or a fragment, or derivative, or a variant thereof. An agent for treating or preventing a neurodegenerative disease, in particular AD, according to the instant invention, may also consist of a nucleotide, an oligonucleotide, or a polynucleotide. Said oligonucleotide or polynucleotide may comprise a nucleotide sequence of the gene coding for CYP11A1, either in sense orientation or in antisense orientation.

In preferred embodiments, the method comprises the application of per se known methods of gene therapy and/or antisense nucleic acid technology to administer said agent or agents. In general, gene therapy includes several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26: 274-278 and Mulligan, Science 1993, 260: 926-931) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, electric shocks). The postnatal gene transfer into the central nervous system has been described in detail (see e.g. Wolff, Curr Opin Neurobiol 1993, 3: 743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN&P 1992, 5: 389-395; Agrawal and Akhtar, Trends Biotechnol 1995, 13: 197-199; Crooke, Biotechnology 1992, 10: 882-6; the contents of which are incorporated herein by reference). Apart from hybridization strategies, the application of ribozymes, i.e. RNA molecules that act as enzymes, destroying RNA that carries the message of disease has also been described (see e.g. Barinaga, Science 1993, 262: 1512-1514). In preferred embodiments, the subject to be treated is a human, and therapeutic antisense nucleic acids or derivatives thereof are directed against transcription products of CYP11A1 gene. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligodeoxynucleotides are known to those of skill in the art (see e.g. Wickstrom, Trends Biotechnol 1992, 10: 281-287; the contents of which are incorporated herein by reference). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy. A recently developed method of regulating the intracellular expression of genes by the use of double-stranded RNA, known variously as RNA interference (RNAi), can be another effective approach for nucleic acid therapy (Hannon, Nature 2002, 418: 244-251).

In further preferred embodiments, the method comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection or liposomal mediated transfection (see Mc Celland and Pardee, Expression Genetics: Accelerated and High-Throughput Methods, Eaton Publishing, Natick, Mass., 1999).

In preferred embodiments, said agent is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector.

Methods of treatment, according to the present invention, comprise the application of therapeutic cloning, transplantation, and stem cell therapy using embryonic stem cells or embryonic germ cells and neuronal adult stem cells, combined with any of the previously described cell- and gene therapeutic methods. Stem cells may be totipotent or pluripotent. They may also be organ-specific. Strategies for repairing diseased and/or damaged brain cells or tissue comprise (i) taking donor cells from an adult tissue. Nuclei of those cells are transplanted into unfertilized egg cells from which the genetic material has been removed. Embryonic stem cells are isolated from the blastocyst stage of the cells which underwent somatic cell nuclear transfer. Use of differentiation factors then leads to a directed development of the stem cells to specialized cell types, preferably neuronal cells (Lanza et al., Nature Medicine 1999, 9: 975-977), or (ii) purifying adult stem cells, isolated from the central nervous system, or from bone marrow (mesenchymal stem cells), for in vitro expansion and subsequent grafting and transplantation, or (iii) directly inducing endogenous neural stem cells to proliferate, migrate, and differentiate into functional neurons (Peterson D A, Curr. Opin. Pharmacol. 2002, 2: 34-42). Adult neural stem cells are of great potential for repairing, damaged or diseased brain tissues, as the germinal centers of the adult brain are free of neuronal damage or dysfunction (Colman A, Drug Discovery World 2001, 7: 66-71).

In preferred embodiments, the subject for treatment or prevention, according to the present invention, can be a human, an experimental animal, e.g. a mouse or a rat, a domestic animal, or a non-human primate. The experimental animal can be an animal model for a neurodegenerative disorder, e.g. a transgenic mouse and/or a knock-out mouse with an AD-type neuropathology.

In a further aspect, the invention features a modulator of an activity, or a level, or both said activity and said level of at least one substance which is selected from the group consisting of (i) the CYP11A1 gene, and/or (ii) a transcription product of the CYP11A1 gene and/or (iii) a translation product of the CYP11A1 gene, and/or (iv) a fragment, or derivative, or variant of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In a further aspect, the invention features a modulator of an activity, or a level, or both said activity and said, level of at least one substance which is selected from the group consisting of (i) the CYP11A1 gene, and/or (ii) a transcription product of the CYP11A1 gene, and/or (iii) a translation product of the CYP11A1 gene, and/or (iv) a fragment, or derivative, or variant of (i) to (iii) for use in a pharmaceutical composition.

In another aspect, the invention provides for the use of a modulator of an activity, or a level, or both said activity and said level of at least one substance which is selected from the group consisting of (i) the CYP11A1 gene, and/or (ii) a transcription product of the CYP11A1 gene and/or (iii) a translation product of the CYP11A1 gene, and/or (iv) a fragment, or derivative, or variant of (i) to (iii) for a preparation of a medicament for treating or preventing a neurodegenerative disease, in particular AD.

In one aspect, the present invention also provides a kit comprising one or more containers filled with a therapeutically or prophylactically effective amount of said pharmaceutical composition.

In a further aspect, the invention features a recombinant, non-human animal comprising a non-native gene sequence coding for CYP11A1, or a fragment, or a derivative, or a variant thereof. The generation of said recombinant, non-human animal comprises (i) providing a gene targeting construct containing said gene sequence and a selectable marker sequence, and (ii) introducing said targeting construct into a stem cell of a non-human animal, and (iii) introducing said non-human animal stem cell into a non-human embryo, and (iv) transplanting said embryo into a pseudopregnant non-human animal, and (v) allowing said embryo to develop to term, and (vi) identifying a genetically altered non-human animal whose genome comprises a modification of said gene sequence in both alleles, and (vii) breeding the genetically altered non-human animal of step (vi) to obtain a genetically altered non-human animal whose genome comprises a modification of said endogenous gene, wherein said gene is mis-expressed, or under-expressed, or over-expressed, and wherein said disruption or alteration results in said non-human animal exhibiting a predisposition to developing symptoms of neuropathology similar to a neurodegenerative disease, in particular AD. Strategies and techniques for the generation and construction of such an animal are known to those of ordinary skill in the art (see e.g. Capecchi, Science 1989, 244: 1288-1292 and Hogan et al., 1994, Manipulating the Mouse Embryo: A Laboratory Manual, Cold Spring Harbor Laboratory Press, Cold Spring Harbor, N.Y. and Jackson and Abbott, Mouse Genetics and Transgenics: A Practical Approach, Oxford University Press, Oxford, England, 1999). It is preferred to make use of such a recombinant non-human animal as an animal model for investigating neurodegenerative diseases, in particular Alzheimer's disease. Such an animal may be useful for screening, testing and validating compounds, agents and modulators in the development of diagnostics and therapeutics to treat neurodegenerative diseases, in particular Alzheimer's disease.

In another aspect, the invention features an assay for screening for a modulator of neurodegenerative diseases, in particular AD, or related diseases and disorders of one or more substances selected from the group consisting of (i) the CYP11A1 gene, and/or (ii) a transcription product of the CYP11A1 gene, and/or (iii) a translation product of the CYP11A1 gene, and/or (iv) a fragment, or derivative, or variant of (i) to (iii). This screening method comprises (a) contacting a cell with a test compound, and (b) measuring the activity, or the level, or both the activity and the level of one or more substances recited in (i) to (iv), and (c) measuring the activity, or the level, or both the activity and the level of said substances in a control cell not contacted with said test compound, and (d) comparing the levels of the substance in the cells of step (b) and (c), wherein an alteration in the activity and/or level of said substances in the contacted cells indicates that the test compound is a modulator of said diseases and disorders.

In one further aspect, the invention features a screening assay for a modulator of neurodegenerative diseases, in particular AD, or related diseases and disorders of one or more substances selected from the group consisting of (i) the CYP11A1 gene, and/or (ii) a transcription product of the CYP11A1 gene, and/or (iii) a translation product of the CYP11A1 gene, and/or (iv) a fragment, or derivative, or variant of (i) to (iii), comprising (a) administering a test compound to a test animal which is predisposed to developing or has already developed symptoms of a neurodegenerative disease or related diseases or disorders, and (b) measuring the activity and/or level of one or more substances recited in (i) to (iv), and (c) measuring the activity and/or level of said substances in a matched control animal which is equally predisposed to developing or has already developed said symptoms and to which animal no such test compound has been administered, and (d) comparing the activity and/or level of the substance in the animals of step (b) and (c), wherein an alteration in the activity and/or level of substances in the test animal indicates that the test compound is a modulator of said diseases and disorders.

In a preferred embodiment, said test animal and/or said control animal is a recombinant, non-human animal which expresses the CYP11A1 gene, or a fragment, or a derivative, or a variant thereof, under the control of a transcriptional regulatory element which is not the native CYP11A1 gene transcriptional control regulatory element.

In another embodiment, the present invention provides a method for producing a medicament comprising the steps of (i) identifying a modulator of neurodegenerative diseases by a method of the aforementioned screening assays and (ii) admixing the modulator with a pharmaceutical carrier. However, said modulator may also be identifiable by other types of screening assays.

In another aspect, the present invention provides for an assay for testing a compound, preferably for screening a plurality of compounds, for inhibition of binding between a ligand and a translation product of the CYP11A1 gene, or a fragment, or derivative, or variant thereof. Said screening assay comprises the steps of (i) adding a liquid suspension of said translation product of the CYP11A1 gene, or a fragment, or derivative, or variant thereof, to a plurality of containers, and (ii) adding a compound or a plurality of compounds to be screened for said inhibition to said plurality of containers, and (iii) adding a detectable, preferably a fluorescently labelled ligand to said containers, and (iv) incubating said translation product of the CYP11A1 gene, or said fragment, or derivative, or variant thereof, and said compound or plurality of compounds, and said detectable, preferably fluorescently labelled ligand, and (v) measuring the amounts of fluorescence associated with said translation product of the CYP11A1 gene, or with said fragment, or derivative, or variant thereof, and (vi) determining the degree of inhibition by one or more of said compounds of binding of said ligand to said translation product of the CYP11A1 gene, or said fragment, or derivative, or variant thereof. It might be preferred to reconstitute said CYP11A1 translation product, or fragment, or derivative, or variant thereof into artificial liposomes to generate the corresponding proteoliposomes to determine the inhibition of binding between a ligand and said CYP11A1 translation product. Methods of reconstitution of CYP11A1 translation products from detergent into liposomes have been detailed (Schwarz et al., Biochemistry 1999, 38: 9456-9464; Krivosheev and Usanov, Biochemistry-Moscow 1997, 62: 1064-1073). Instead of utilizing a fluorescently labelled ligand, it might in some aspects be preferred to use any other detectable label known to the person skilled in the art, e.g. radioactive labels, and detect it accordingly. Said method may be useful for the identification of novel compounds as well as for evaluating compounds which have been improved or otherwise optimized in their ability to inhibit the binding of a ligand to a translation product of the CYP11A1 gene, or a fragment, or derivative, or variant thereof. One example of a fluorescent binding assay, in this case based on the use of carrier particles, is disclosed and described in patent application WO 00/52451. A further example is the competitive assay method as described in patent WO 02/01226. Preferred signal detection methods for screening assays of the instant invention are described in the following patent applications: WO 96/13744, WO 98/16814, WO 98/23942, WO 99/17086, WO 99/34195, WO 00/66985, WO 01/59436, WO 01/59416.

In one further embodiment, the present invention provides a method for producing a medicament comprising the steps of (i) identifying a compound as an inhibitor of binding between a ligand and a translation product of the CYP11A1 gene by the aforementioned inhibitory binding assay and (ii) admixing the compound with a pharmaceutical carrier. However, said compound may also be identifiable by other types of screening assays.

In another aspect, the invention features an assay for testing a compound, preferably for screening a plurality of compounds to determine the degree of binding of said compounds to a translation product of the CYP11A1 gene, or to a fragment, or derivative, or variant thereof. Said screening assay comprises (i) adding a liquid suspension of said translation product of the CYP11A1 gene, or a fragment, or derivative, or variant thereof, to a plurality of containers, and (ii) adding a detectable, preferably a fluorescently labelled compound or a plurality of detectable, prferably fluorescently labelled compounds to be screened for said binding to said plurality of containers, and (iii) incubating said translation product of the CYP11A1 gene, or said fragment, or derivative, or variant thereof, and said detectable, preferably fluorescently labelled compound or detectable, preferably fluorescently labelled compounds, and (iv) measuring the amounts of fluorescence associated with said translation product of the CYP11A1 gene, or with said fragment, or derivative, or variant thereof, and (v) determining the degree of binding by one or more of said compounds to said translation product of the CYP11A1 gene, or said fragment, or derivative, or variant thereof. In this type of assay it might be preferred to use a fluorescent label. However, any other type of detectable label might also be employed. Said assay methods may be useful for the identification of novel compounds as well as for evaluating compounds which have been improved or otherwise optimized in their ability to bind to said translation product of the CYP11A1 gene, or a fragment, or derivative, or variant thereof.

In one further embodiment, the present invention provides a method for producing a medicament comprising the steps of (i) identifying a compound as a binder to a gene product of the CYP11A1 gene by the aforementioned binding assays and (ii) admixing the compound with a pharmaceutical carrier. However, said compound may also be identifiable by other types of screening assays.

In another embodiment, the present invention provides for a medicament obtainable by any of the methods according to the herein claimed screening assays. In one further embodiment, the instant invention provides for a medicament obtained by any of the methods according to the herein claimed screening assays.

The present invention features a protein molecule shown in SEQ ID NO. 1, said protein molecule being a translation product of the CYP11A1 gene, or a fragment, or derivative, or variant thereof, for use as a diagnostic target for detecting a neurodegenerative disease, preferably Alzheimer's disease.

The present invention also features a protein molecule shown in SEQ ID NO. 1, said protein molecule being a translation product of the CYP11A1 gene, or a fragment, or derivative, or variant thereof, for use as a screening target for reagents or compounds preventing, or treating, or ameliorating a neurodegenerative disease, preferably Alzheimer's disease.

The present invention features an antibody which is specifically immunoreactive with an immunogen, wherein said immunogen is a translation product of the CYP11A1 gene or a fragment, or derivative, or variant thereof. The immunogen may comprise immunogenic or antigenic epitopes or portions of a translation product of said gene, wherein said immunogenic or antigenic portion of a translation product is a polypeptide, and wherein said polypeptide elicits an antibody response in an animal, and wherein said polypeptide is immunospecifically bound by said antibody. Methods for generating antibodies are well known in the art (see Harlow et al., Antibodies, A Laboratory Manual, Cold Spring Harbor Laboratory Press, Cold Spring Harbor, N.Y., 1988). The term “antibody”, as employed in the present invention, encompasses all forms of antibodies known in the art, such as polyclonal, monoclonal, chimeric, recombinatorial, anti-idiotypic, humanized, or single chain antibodies, as well as fragments thereof (see Dubel and Breitling, Recombinant Antibodies, Wiley-Liss, New York, N.Y., 1999). Antibodies of the present invention are useful, for instance, in a variety of diagnostic and therapeutic methods, based on state-in-the-art techniques (see Harlow and Lane, Using Antibodies: A Laboratory Manual, Cold Spring Harbor Laboratory Press, Cold Spring Harbor, N.Y., 1999 and Edwards R., Immunodiagnostics: A Practical Approach, Oxford University Press, Oxford, England, 1999) such as enzyme-immuno assays (e.g. enzyme-linked immunosorbent assay, ELISA), radioimmuno assays, chemoluminescence-immuno assays, Western-blot, immunoprecipitation and antibody microarrays. These methods involve the detection of translation products of the CYP11A1 gene, or fragments, or derivatives, or variants thereof.

In a preferred embodiment of the present invention, said antibodies can be used for detecting the pathological state of a cell in a sample from a subject, comprising immunocytochemical staining of said cell with said antibody, wherein an altered degree of staining, or an altered staining pattern in said cell compared to a cell representing a known health status indicates a pathological state of said cell. Preferably, the pathological state relates to a neurodegenerative disease, in particular to AD. Immunocytochemical staining of a cell can be carried out by a number of different experimental methods well known in the art. It might be preferred, however, to apply an automated method for the detection of antibody binding, wherein the determination of the degree of staining of a cell, or the determination of the cellular or subcellular staining pattern of a cell, or the topological distribution of an antigen on the cell surface or among organelles and other subcellular structures within the cell, are carried out according to the method described in U.S. Pat. No. 6,150,173.

Other features and advantages of the invention will be apparent from the following description of figures and examples which are illustrative only and not intended to limit the remainder of the disclosure in any way.

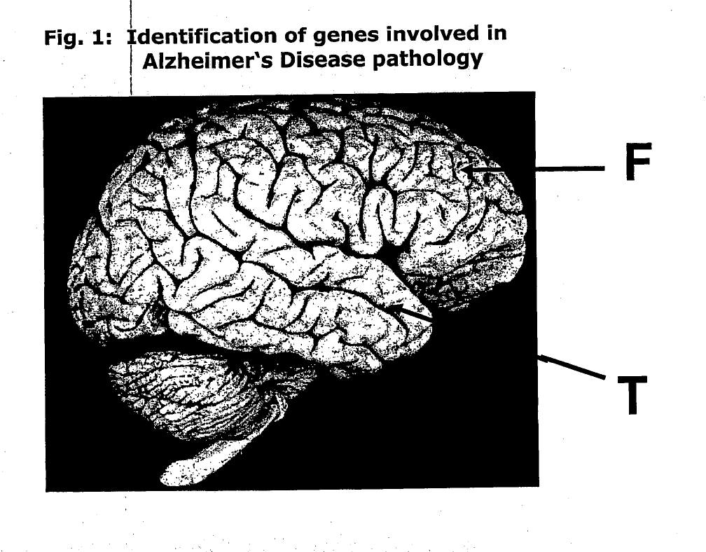

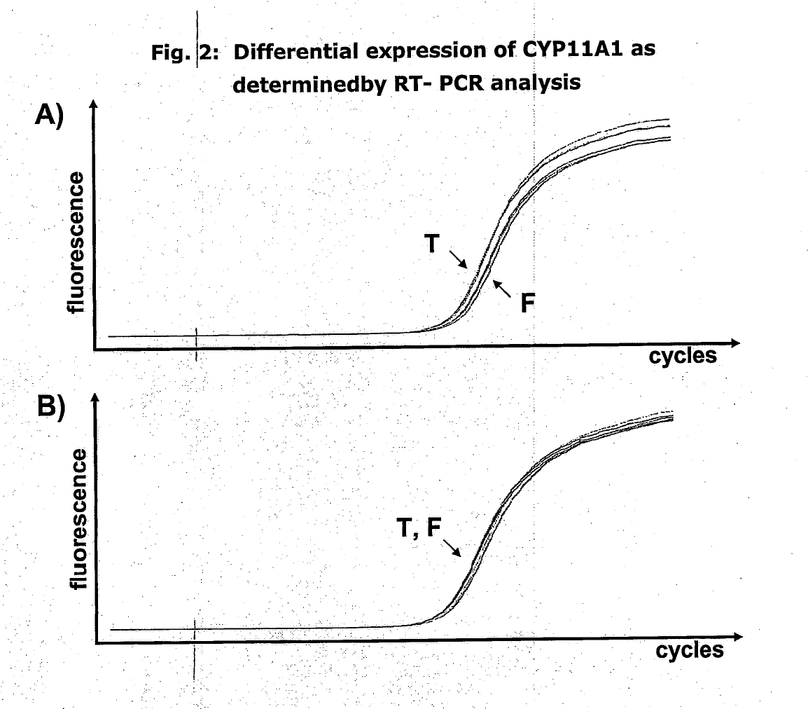

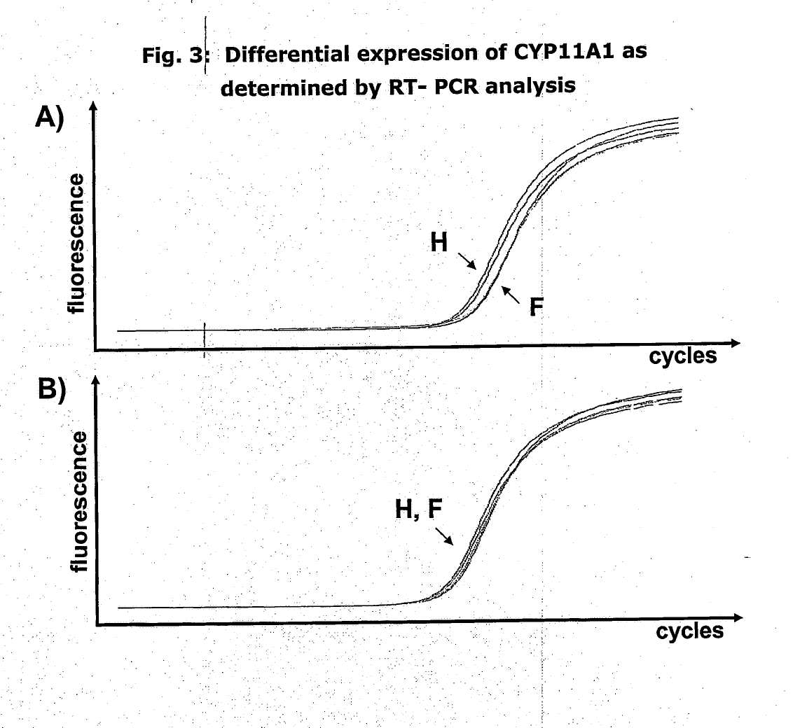

FIG. 1 depicts the brain regions with selective vulnerability to neuronal loss and degeneration in AD. Primarily, neurons within the inferior temporal lobe, the entorhinal cortex, the hippocampus, and the amygdala are subject to degenerative processes in AD (Terry et al., Annals of Neurology 1981, 10:184-192). These brain regions are mostly involved in the processing of learning and memory functions. In contrast, neurons within the frontal cortex, the occipital cortex, and the cerebellum remain largely intact and preserved from neurodegenerative processes in AD. Brain tissues from the frontal cortex (F), the temporal cortex (T), and the hippocampus (H) of AD patients and healthy, age-matched control individuals were used for the herein disclosed examples. For illustrative purposes, the image of a normal healthy brain was taken from a publication by Strange (Brain Biochemistry and Brain Disorders, Oxford University Press, Oxford, 1992, p.4). FIGS. 2 and 3 demonstrate the differential expression of the CYP11A1 gene in AD brain tissues by quantitative RT-PCR analysis. Quantification of RT-PCR products from RNA samples collected from the frontal cortex (F) and the temporal cortex (T) of AD patients (FIG. 2a) and samples from the frontal cortex (F) and the hippocampus (H) of AD patients (FIG. 3a) was performed by the LightCycler rapid thermal cycling technique. Likewise, samples of healthy, age-matched control individuals were compared (FIG. 2b for frontal cortex and temporal cortex, FIG. 3b for frontal cortex and hippocampus). The data were normalized to the combined average values of a set of standard genes which showed no significant differences in their gene expression levels. Said set of standard genes consisted of genes for cyclophilin B, the ribosomal protein S9, the transferrin receptor, GAPDH, and beta-actin. The figures depict the kinetics of amplification by plotting the cycle number against the amount of amplified material as measured by its fluorescence. Note that the amplification kinetics of the CYP11A1 cDNAs from both, the frontal and temporal cortices of a normal control individual, and from the frontal cortex and hippocampus of a normal control individual, respectively, during the exponential phase of the reaction are juxtaposed (FIGS. 2b and 3b, arrowheads), whereas in Alzheimer's disease (FIGS. 2a and 3a, arrowheads) there is a significant separation of the corresponding curves, indicating a differential expression of the CYP11A1 gene in the respective analyzed brain regions.

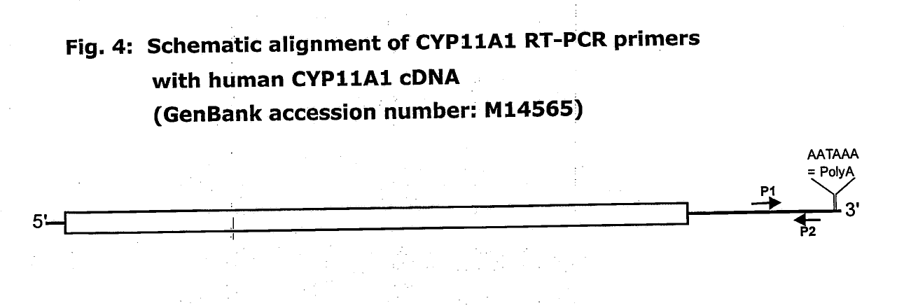

FIG. 4 charts the schematic alignment of the CYP11A1 primer sequences to the nucleotide sequence of the human CYP11A1 gene (Chung et al., Proc. Natl. Acad. Sci. U.S.A. 1986, 83: 8962-8966; GenBank accession number M14565); polyA: polyadenylation site; primer positions P1, P2 are indicated by arrows; the open box represents the CYP11A1 open reading frame (positions 45-1610).

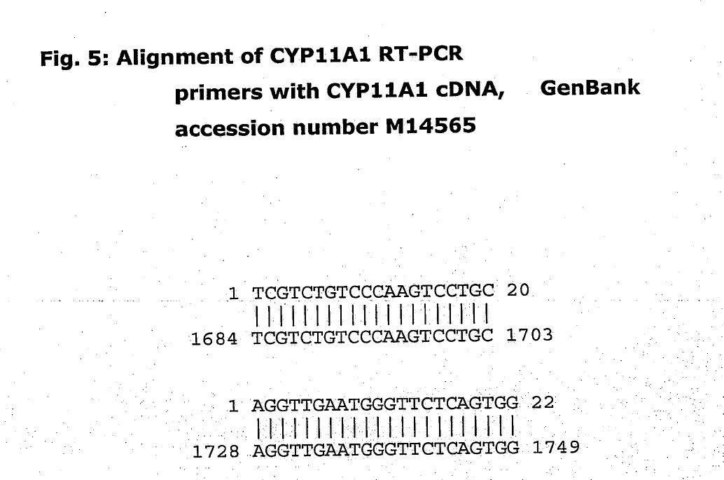

FIG. 5 outlines the exact sequence alignments of the CYP11A1 primer pair to the nucleotide sequence of the human CYP11A1 gene (Chung et al., Proc. Natl. Acad. Sci. U.S.A. 1986, 83: 8962-8966; GenBank accession number M14565).

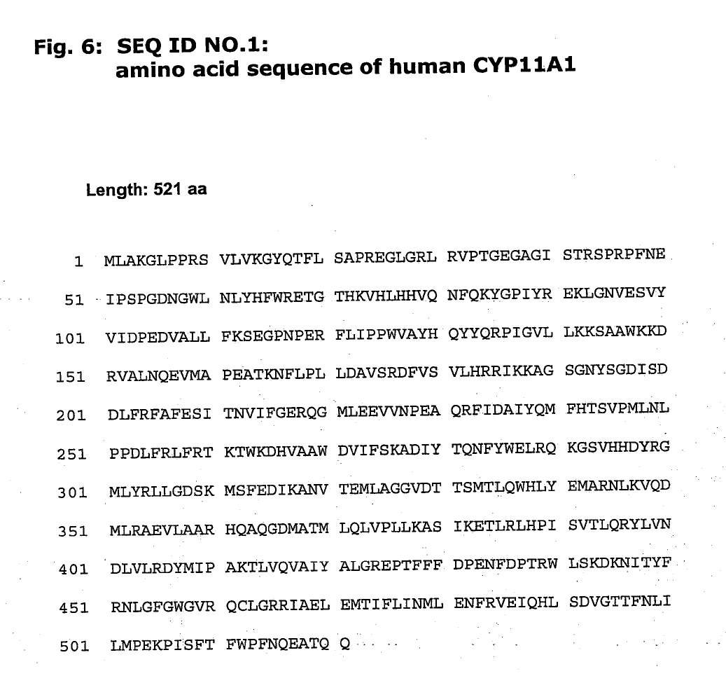

FIG. 6 discloses SEQ ID NO. 1, the polypeptide sequence of human CYP11A1, comprising 521 amino acids (GenBank accession number P05108).

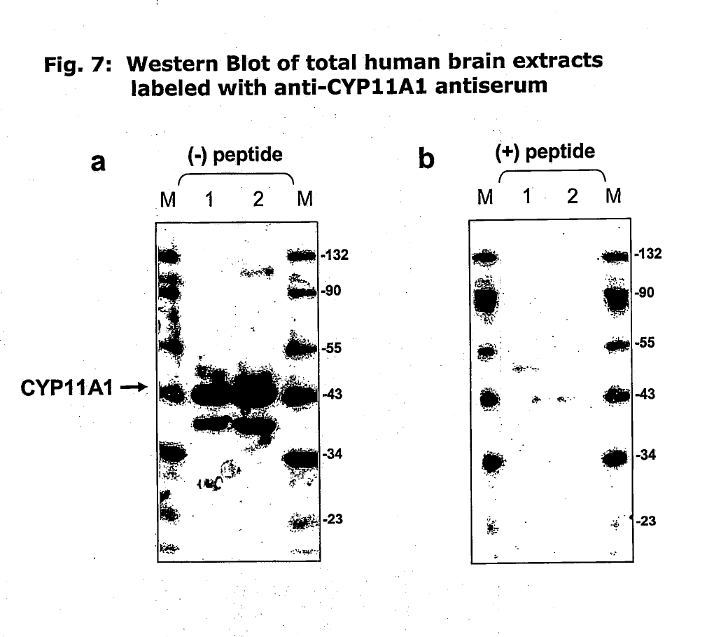

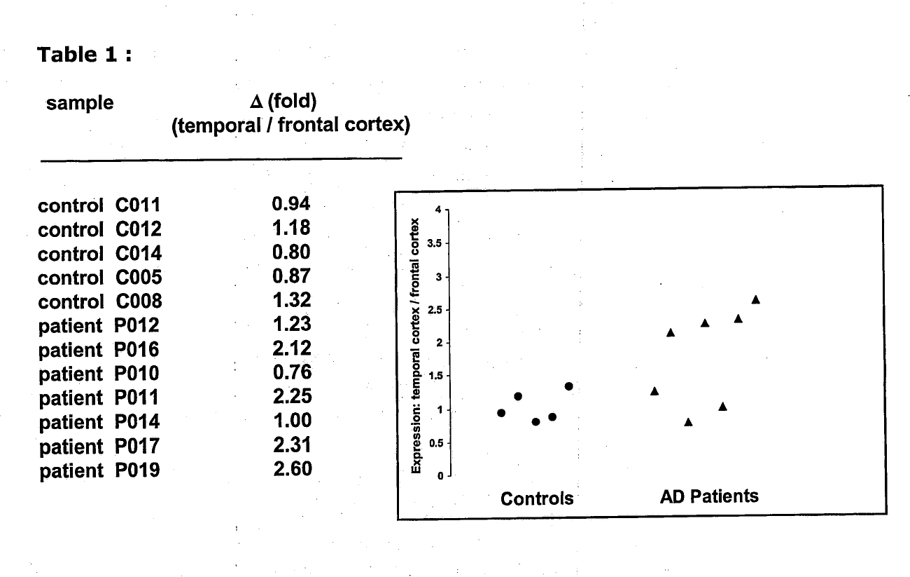

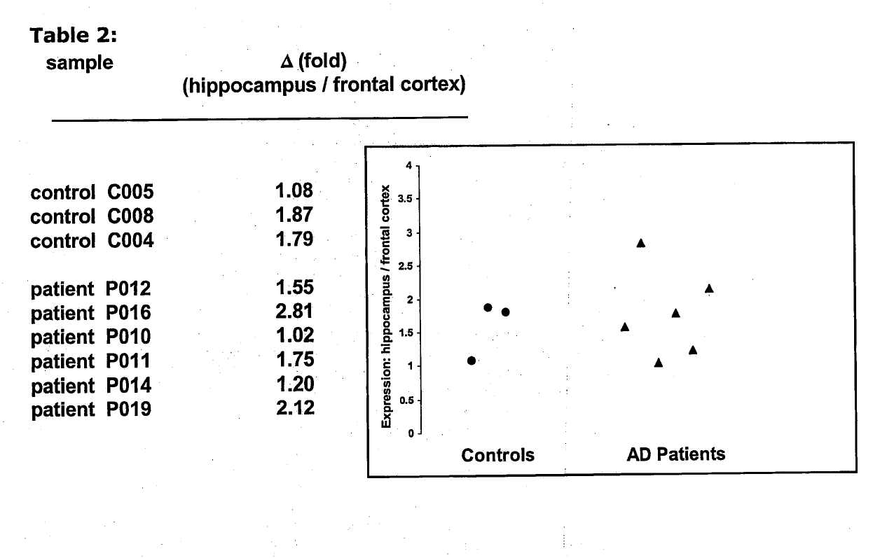

FIG. 7 depicts a Western blot image of total human brain extracts labeled with an affinity-purified rabbit anti-CYP11A1 antiserum raised against a peptide corresponding to amino acids 179 to 194 of SEQ ID NO. 1.

Lane 1 and 2: total brain extracts of two different human brain samples (FIG. 7a and 7b). The arrow indicates a major band just above 43 kDa (FIG. 7a), a size less than the predicted 61 kDa of the CYP11A1 mitochondrial precusor and of the 57 kDa mature protein, which is due to an apparent faster electrophoretic mobility of the CYP11A1 protein. This mobility shift has been published also for rat CYP11A1, showing an apparent molecular size of 49 kDa. The lower main band on the blot is predicted to be the mature human CYP11A1. All immunoreactive bands could be competed by preincubation of the antiserum with the corresponding peptide (FIG. 7b).

Table 1 lists the gene expression levels in the temporal cortex relative to the frontal cortex for the CYP11A1 gene in seven Alzheimer's disease patients, herein identified by internal reference numbers P010, P011, P012, P014, P016, P017, P019 (0.76 to 2.60 fold) and five healthy, age-matched control individuals, herein identified by internal reference numbers C005, C008, C011, C012, C014 (0.80 to 1.32 fold). The scatter plot diagram visualizes individual values of the temporal to frontal cortex regulation ratios in control samples (dots) and in AD patient samples (triangles), respectively.

Table 2 lists the CYP11A1 gene expression levels in the hippocampus relative to the frontal cortex in six Alzheimer's disease patients, herein identified by internal reference numbers P10, P011, P012, P014, P016, P019 (1.02 to 2.81 fold) and three healthy, age-matched control individuals, herein identified by internal reference numbers C004, C005, C008 (1.08 to 1.87 fold). The scatter plot diagram visualizes individual values of the hippocampus to frontal cortex regulation ratios in control samples (dots) and in AD patient samples (triangles), respectively.

### EXAMPLE I

(i) Brain tissue dissection from patients with AD:

Brain tissues from AD patients and age-matched control subjects were collected within 6 hours post-mortem and immediately frozen on dry ice. Sample sections from each tissue were fixed in paraformaldehyde for histopathological confirmation of the diagnosis. Brain areas for differential expression analysis were identified (see FIG. 1) and stored at −80° C. until RNA extractions were performed.

(ii) Isolation of total mRNA:

Total RNA was extracted from post-mortem brain tissue by using the RNeasy kit (Qiagen) according to the manufacturer's protocol. The accurate RNA concentration and the RNA quality were determined with the DNA LabChip system using the Agilent 2100 Bioanalyzer (Agilent Technologies). For additional quality testing of the prepared RNA, i.e. exclusion of partial degradation and testing for DNA contamination, specifically designed intronic GAPDH oligonucleotides and genomic DNA as reference control were used to generate a melting curve with the LightCycler technology as described in the manufacturer's protocol (Roche).

(iii) Quantitative RT-PCR analysis:

The expression levels of the human CYP11A1 gene in temporal versus frontal cortex were analyzed using the LightCycler technology (Roche). This technique features rapid thermal cyling for the polymerase chain reaction as well as real-time measurement of fluorescent signals during amplification and therefore allows for highly accurate quantification of RT-PCR products by using a kinetic, rather than an endpoint readout. The ratios of CYP11A1 cDNA from the temporal cortex and frontal cortex, and from the hippocampus and frontal cortex, respectively, were determined (relative quantification).

First, a standard curve was generated to determine the efficiency of the PCR with specific primers for human CYP11A1 (5′-TCGTCTGTCCCAAGTCCTGC-3′ and 5′-CCACTGAGAACCCATTCAACCT-3′). PCR amplification (95° C. and 1 sec, 56° C. and 5 sec, and 72° C. and 5 sec) was performed in a volume of 20 μl containing LightCycler-FastStart DNA Master SYBR Green I mix (contains FastStart Taq DNA polymerase, reaction buffer, dNTP mix with dUTP instead of dTTP, SYBR Green I dye, and 1 mM MgCl₂; Roche), 0.5 μM primers, 2 μl of a cDNA dilution series (final concentration of 40, 20, 10, 5, 1 and 0.5 ng human total brain cDNA; Clontech) and, depending on the primers used, additional 3 mM MgCl₂. Melting curve analysis revealed a single peak at approximately 84° C. with no visible primer dimers. Quality and size of the PCR product were determined with the DNA LabChip system (Agilent 2100 Bioanalyzer, Agilent Technologies). A single peak at the expected size of 66 bp for CYP11A1 was observed in the electropherogram of the sample.

In an analogous manner, the PCR protocol was applied to determine the PCR efficiency of a set of reference genes which were selected as a reference standard for quantification. In the present invention, the mean value of five such reference genes was determined: (1) cyclophilin B, using the specific primers 5′-ACTGAAGCACTACGGGCCTG-3′ and 5′-AGCCGTTGGTGTCTTTGCC-3′ except for MgCl₂ (an additional 1 mM was added instead of 3 mM). Melting curve analysis revealed a single peak at approximately 87° C. with no visible primer dimers. Agarose gel analysis of the PCR product showed one single band of the expected size (62 bp). (2) Ribosomal protein S9 (RPS9), using the specific primers 5′-GGTCAAATTTACCCTGGCCA-3′ and 5′-TCTCATCAAGCGTCAGCAGTTC-3′ (exception: additional 1 mM MgCl₂ was added instead of 3 mM). Melting curve analysis revealed a single peak at approximately 85° C. with no visible primer dimers. Agarose gel analysis of the PCR product showed one single band with the expected size (62 bp). (3) beta-actin, using the specific primers 5′-TGGAACGGTGAAGGTGACA-3′ and 5′-GGCAAGGGACTTCCTGTAA-3′. Melting curve analysis revealed a single peak at approximately 87° C. with no visible primer dimers. Agarose gel analysis of the PCR product showed one single band with the expected size (142 bp). (4) GAPDH, using the specific primers 5′-CGTCATGGGTGTGAACCATG-3′ and 5′-GCTAAGCAGTTGGTGGTGCAG-3′. Melting curve analysis revealed a single peak at approximately 83° C. with no visible primer dimers. Agarose gel analysis of the PCR product showed one single band with the expected size (81 bp). (5) Transferrin receptor TRR, using the specific primers 5′-GTCGCTGGTCAGTTCGTGATT-3′ and 5′-AGCAGTTGGCTGTTGTACCTCTC-3′. Melting curve analysis revealed a single peak at approximately 83° C. with no visible primer dimers. Agarose gel analysis of the PCR product showed one single band with the expected size (80 bp).

For calculation of the values, first the logarithm of the cDNA concentration was plotted against the threshold cycle number Cₜ for CYP11A1 and the five reference standard genes. The slopes and the intercepts of the standard curves (i.e. linear regressions) were calculated for all genes. In a second step, cDNAs from temporal cortex and frontal cortex, and from hippocampus and frontal cortex, respectively, were analyzed in parallel and normalized to cyclophilin B. The Cₜ values were measured and converted to ng total brain cDNA using the corresponding standard curves:
10ˆ(Cₜvalue−intercept)/slope) (ng total brain-cDNA]

The values for frontal and temporal cortex cDNAs of CYP11A1, and the values for hippocampus and frontal cortex CYP11A1 cDNAs, respectively, were normalized to cyclophilin B and the ratios were calculated according to formulas:

$$Ratio = \frac{CYP 11 A 1 temporal [ ng ] / cyclophilin B temporal [ ng ]}{CYP 11 A 1 frontal [ ng ] / cyclophilin B frontal [ ng ]}$$

$$Ratio = \frac{\begin{matrix}CYP 11 A 1 hippocampus [ ng ] / \\ cyclophilin B hippocampus [ ng ]\end{matrix}}{CYP 11 A 1 frontal [ ng ] / cyclophilin B frontal [ ng ]}$$

In a third step, the set of reference standard genes was analyzed in parallel to determine the mean average value of the temporal to frontal ratios, and of the hippocampal to frontal ratios, respectively, of expression levels of the reference standard genes for each individual brain sample. As cyclophilin B was analyzed in step 2 and step 3, and the ratio from one gene to another gene remained constant in different runs, it was possible to normalize the values for CYP11A1 to the mean average value of the set of reference standard genes instead of normalizing to one single gene alone. The calculation was performed by dividing the respective ratio shown above by the deviation of cyclophilin B from the mean value of all housekeeping genes. The results of such quantitative RT-PCR analysis for the CYP11A1 gene are shown in FIGS. 2 and 3.

(iv) Immunoblotting:

Brain tissue (0.1 g) was homogenized in 1 ml RIPA buffer (150 mM sodium chloride, 50 mM tris-HCl, pH7.4, 1 mM ethylenediamine-tetraacetic acid, 1 mM phenylmethylsulfonyl flouride, 1% Triton X-100, 1% sodium deoxycholic acid, 1% sodium dodecylsulfate, 5 μg/ml of aprotinin, 5 μg/ml of leupeptin) on ice. After centrifuging for 15 min at 13,000 rpm, the supernatant was diluted five-fold in SDS-loading buffer. Aliquots of 20 μl of the diluted sample were resolved by SDS-PAGE (12% polyacrylamide) and transferred to PVDF Western Blotting membranes (Boehringer Mannheim). The blots, preincubated with or without 2 μM of the corresponding peptide for 30 min at room temperature, were labeled with affinity-purified rabbit anti-CYP11A1 antiserum (custom-made, Davids Biotechnology, Regensburg) and developed with the ECL chemoluminescence detection kit (Amersham Pharmacia).

1. A method of diagnosing or prognosticating a neurodegenerative disease, in a subject, or determining whether a subject is at increased risk of developing said disease, comprising determining a level and/or an activity of
(i) a transcription product of the CYP11A1 gene, and/or
(ii) a translation product of the CYP11A1 gene, and/or
(iii) a fragment, or derivative, or variant of said transcription or translation product,
in a sample obtained from said subject and comparing said level and/or said activity to a reference value representing a known disease or health status, thereby diagnosing or prognosticating said neurodegenerative disease in said subject, or determining whether said subject is at increased risk of developing said neurodegenerative disease.

2. A kit for diagnosing or prognosticating a neurodegenerative disease in a subject, or determining the propensity or predisposition of a subject to develop such a disease by the steps of:
(i) detecting in a sample obtained from said subject a level, or an activity, or both said level and said activity of a transcription product and/or of a translation product of a gene coding for CYP11A1, and
(ii) comparing said level or activity, or both said level and said activity of a transcription product and/or of a translation product of a gene coding for CYP11A1 to a reference value representing a known health status and/or to a reference value representing a known disease status, and said level, or activity, or both said level and said activity, of said transcription product and/or said translation product is varied compared to a reference value representing a known health status, and/or is similar or equal to a reference value representing a known disease status, said kit comprising:
at least one reagent which is selected from the group consisting of (i) reagents that selectively detect a transcription product of a gene coding for CYP11A1 and (ii) reagents that selectively detect a translation product of a gene coding for CYP11A1.

3. A modulator of an activity and/or of a level of at least one substance which is selected from the group consisting of
(i) the CYP11A1 gene,
(ii) a transcription product of the CYP11A1 gene,
(iii) a translation product of the CYP11A1 gene, and
(iv) a fragment, or derivative, or variant of (i) to (iii).

4. A recombinant, non-human animal comprising a non-native gene sequence coding for CYP11A1 or a fragment, or a derivative, or a variant thereof, said animal being obtainable by:
(i) providing a gene targeting construct comprising said gene sequence and a selectable marker sequence, and
(ii) introducing said targeting construct into a stem cell of a non-human animal, and
(iii) introducing said non-human animal stem cell into a non-human embryo, and
(iv) transplanting said embryo into a pseudopregnant non-human animal, and
(v) allowing said embryo to develop to term, and
(vi) identifying a genetically altered non-human animal whose genome comprises a modification of said gene sequence in both alleles, and
(vii) breeding the genetically altered non-human animal of step (vi) to obtain a genetically altered non-human animal whose genome comprises a modification of said endogenous gene, wherein said disruption results in said non-human animal exhibiting a predisposition to developing symptoms of a neurodegenerative disease or related diseases or disorders.

5. A method for screening for a modulator of neurodegenerative diseases, or related diseases or disorders of one or more substances selected from the group consisting of
(i) the CYP11A1 gene,
(ii) a transcription product of the CYP11A1 gene,
(iii) a translation product of the CYP11A1 gene, and
(iv) a fragment, or derivative, or variant of (i) to (iii), said method comprising:
(a) contacting a cell with a test compound;
(b) measuring the activity and/or level of one or more substances recited in (i) to (iv);
(c) measuring the activity and/or level of one or more substances recited in (i) to (iv) in a control cell not contacted with said test compound; and
(d) comparing the levels and/or activities of the substance in the cells of step (b) and (c), wherein an alteration in the activity and/or level of substances in the contacted cells indicates that the test compound is a modulator of said diseases or disorders.

6. A method of screening for a modulator of neurodegenerative diseases, or related diseases or disorders of one or more substances selected from the group consisting of
(i) the CYP11A1 gene,
(ii) a transcription product of the CYP11A1 gene,
(iii) a translation product of the CYP11A1 gene, and
(v) a fragment, or derivative, or variant of (i) to (iii),
said method comprising:
(a) administering a test compound to a test animal which is predisposed to developing or has already developed symptoms of a neurodegenerative disease or related diseases or disorders in respect of the substances recited in (i) to (iv);
(b) measuring the activity and/or level of one or more substances recited in (i) to (iv);
(c) measuring the activity and/or level of one or more substances recited in (i) or (iv) in a matched control animal which is predisposed to developing or has already developed symptoms of a neurodegenerative disease or related diseases or disorders in respect to the substances recited in (i) to (iv) and to which animal no such test compound has been administered;
(d) comparing the activity and/or level of the substance in the animals of step (b) and (c), wherein an alteration in the activity and/or level of substances in the test animal indicates that the test compound is a modulator of said diseases or disorders.

7. The method according to claim 6 wherein said test animal and/or said control animal is a recombinant animal which expresses the CYP11A1 gene, or a fragment, or a variant, or a derivative thereof, under the control of a transcriptional control element which is not the native CYP11A1 gene transcriptional control element.

8. An assay for testing a compound, or a plurality of compounds to determine the degree of binding of said compounds to a translation product of the CYP11A1 gene, or to a fragment, or derivative, or variant thereof, said assay comprising the steps of:
(i) adding a liquid suspension of said CYP11A1 translation product, or a fragment, or derivative, or variant thereof, to a plurality of containers;
(ii) adding a detectable, labelled compound or a plurality of detectable, labelled compounds to be screened for said binding to said plurality of containers;
(iii) incubating said CYP11A1 translation product, or said fragment, or derivative, or variant thereof, and said detectable, labelled compound or compounds;
(iv) measuring amounts of detectable compound or detectable label associated with said CYP11A1 translation product, or with said fragment, or derivative, or variant thereof; and
(v) determining the degree of binding by one or more of said compounds to said CYP11A1 translation product, or said fragment, or derivative, or variant thereof.

9. The method of claim 1, comprising determining a level and/or an activity of a protein molecule of SEQ ID NO. 1, said protein molecule being a translation product of the gene coding for CYP11A1, or a fragment, or derivative, or variant thereof.

10. The method of claim 5, wherein said screening is for a modulator of a protein molecule of SEQ ID NO. 1, said protein molecule being a translation product of the gene coding for CYP11A1, or a fragment, or derivative, or variant thereof, wherein said modulator is a reagent or compound for preventing, or treating, or ameliorating a neurodegenerative disease.

11. A method of detecting a pathological state of a cell in a sample obtained from a subject, comprising immunocytochemical staining of said cell with an antibody specifically immunoreactive with an immunogen, wherein said immunogen is a translation product of the gene coding for CYP11A1, or a fragment, or derivative, or variant thereof, wherein an altered degree of staining, or an altered staining pattern in said cell compared to a cell representing a known health status indicates a pathological state of said cell.

12. The method of claim 1, wherein said neurodegenerative disease is Alzheimer's disease.

13. The kit of claim 2, wherein said neurodegenerative disease is Alzheimer's disease.

14. The method of claim 5, wherein said neurodegenerative disease is Alzheimer's disease.

15. The method of claim 6, wherein said neurodegenerative disease is Alzheimer's disease.

16. The assay of claim 8, wherein said detectable, labelled compound or compounds are fluorescently labelled compound or compounds.

17. The assay of claim 8, wherein the detectable label is fluorescence.

18. The method of claim 9, wherein said neurodegenerative disease is Alzheimer's disease.

19. The method of claim 10, wherein said neurodegenerative disease is Alzheimer's disease.

20. The method of claim 6, wherein said screening is for a modulator of a protein molecule of SEQ ID NO. 1, said protein molecule being a translation product of the gene coding for CYP11A1, or a fragment, or derivative, or variant thereof, wherein said modulator is a reagent or compound for preventing, or treating, or ameliorating a neurodegenerative disease.

21. The method of claim 20, wherein said neurodegenerative disease is Alzheimer's disease.
